(19) United States
(12) Patent Application Publication
(10) Pub. No.: US 2002/0192201 A1
(43) Pub. Date: Dec. 19, 2002

(54) Crohn's disease treatment methods

(76) Inventor: Ira Shafran, Winter Park, FL (US)

Correspondence Address: Jacqueline E. Hartt, Allen, Dyer, Doppelt, Milbrath & Gilchrist, P.A., 255 South Orange Avenue, Suite 1401, P.O. Box 3791, Orlando, FL, 32802-3791, US

(21) Appl. No.: 10/165,034

(22) Filed: Jun. 7, 2002

Related U.S. Application Data
(63) Continuation-in-part of application No. 09/968,681, filed on Oct. 1, 2001.
Application No. 09/968,681 is a continuation-in-part of application No. 09/404,095, filed on Sep. 23, 1999, now Pat. No. 6,297,015.
(60) Provisional application No. 60/101,579, filed on Sep. 24, 1998.

(51) Int. Cl. A61K 45/00; A61K 31/7048; A61K 31/496

(52) U.S. Cl. 424/93.43; 514/28; 514/252.13

(57) ABSTRACT

A method for treating a human patient includes screening for Crohn's disease by simultaneously contacting a human serum sample with an antigen composition comprising a 35 kD protein expressed by a recombinant p35 clone specific to sera from Johne's disease and a 36 kD protein expressed by a recombinant p36 clone specific to sera from Crohn's disease. A bound antibody-antigen complex to the antigen composition is detected, the bound antibody-antigen complex detecting a presence of Mycobacterium avium ss. paratuberculosis (MAP). If the screening results are positive, the patient is administered a regimen of an antibiotic effective in and sufficient for eradicating a presence of MAP. Preferably a probiotic and specific carbohydrate diet are also administered. In a related method Crohn's disease is screened for by performing an ELISA analysis for serum antibodies to MAP, and, for patients screening positive for MAP, the antibiotic regimen is administered.

### CROSS-REFERENCE TO RELATED APPLICATIONS

This application is a continuation-in-part of application Ser. No. 09/968,681, “Crohn's Disease Treatment Methods,” filed Oct. 1, 2001, which is a continuation-in part of application Ser. No. 09/404,095, “Crohn's Disease Diagnostic Treatment Methods and Compositions, now U.S. Pat. No. 6,297,015, which in turn claims priority from provisional application “Crohn's Disease Diagnostic Method and Composition,” Serial No. 60/101,579, filed Sep. 24, 1998, all of which are commonly owned with the present invention and which are incorporated herein by reference.

### BACKGROUND OF THE INVENTION

1. Field of the Invention

The present invention relates to compositions and methods for diagnosing and treating Crohn's disease, and, more particularly, to such compositions and methods for screening for a presence of a bacterium believed involved in causing Crohn's disease and for treating patients shown by the screening method to be infected with the bacterium.

2. Description of Related Art

Crohn's disease is an inflammatory bowel disease that affects 2-3 million Americans, with a typical onset between 15 and 25 years of age. Crohn's is a chronic disorder that causes inflammation or ulceration in the small and/or large intestine, extending into the deeper layers of the intestinal wall. Sometimes the inflammation may also affect the mouth, esophagus, stomach, duodenum, appendix, or anus. Although Crohn's is a chronic condition, periods of remission may occur, with recurrences unpredictable. Two forms of the disease, perforating and nonperforating, are believed to occur.

Common symptoms of Crohn's disease include abdominal pain and diarrhea. There may also be rectal bleeding, weight loss, and fever. The bleeding may be serious and persistent, leading to anemia. Children may suffer delayed development and stunted growth.

Current diagnoses are performed by blood test to detect anemia and elevated white blood cell count, colon biopsy, and lower gastrointestinal x-ray series.

There are drugs that can be helpful in controlling Crohn's disease, but at present there is no cure. Treatment is aimed at correcting nutritional deficiencies, controlling inflammation, relieving the symptoms of abdominal pain, diarrhea, and rectal bleeding. Drugs known to be used for this condition can help, but side effects can be deleterious. Surgeries that may be performed to alleviate symptoms include the removal of inflamed areas, draining of abscesses, and bowel resection.

The cause of Crohn's disease has been debated since its recognition in the early part of the twentieth century. There are those who believe that at least some of the cases are caused by a bacterium, specifically Mycobacterium paratuberculosis, which is endemic in foods derived from cattle and water supplies in the Western world. Crohn's patients have been reported to have been cured by an antibiotic or a multidrug antibiotic regime having activity against that organism.

Mycobacterium paratuberculosis is an obligate pathogen; that is, it cannot multiply outside the cells of animals. It is known to be present in a wide variety of animals, including primates and humans. The best-studied animal paratuberculosis is bovine Johne's disease (BJD), a disease that causes chronic diarrhea, weight loss, and malnutrition in cattle and affects up to 25% of the dairy cattle in the United States. Cows infected with BJD are known to secrete Mycobacterium paratuberculosis in their milk, which is not destroyed by standard milk pasteurization methods, but only by ultrapasteurization. This bacterium has also been cultured from a municipal water supply in the United States.

One can be exposed to Mycobacterium paratuberculosis and not develop Crohn's disease, if the immune system is capable of fighting the bacterium, resulting only in a transient intestinal infection with no after-effects. However, those susceptible to inflammatory bowel disease, including those with a genetic predisposition or being immunosuppressed, can develop the disease.

Mycobacterium paratuberculosis occurs in two forms, the bacillary form and the spheroplast form, in which no cell wall is present. The former, which may be required to cause disease, is easily detected in animals by a simple chemical test; the latter, however, has only been found to be detectable with genetic testing techniques, such as polymerase chain reaction (PCR), to detect the 1451-bp IS900 insertion sequence unique to Mycobacterium paratuberculosis. PCR methods, however, can fail under conditions in which the amount of spheroplast present in the tissue is low or when tissue preserved in wax-embedded paraffin blocks is used. Another method is direct culturing of the organism followed by IS900 detection.

Currently available serological tests for Johne's disease are believed to have poor sensitivity and specificity, and fecal smear microscopy and fecal culture in early stages of infection are of limited value. EI-Zaatari et al. (Current Microbiol. 29, 177-184, 1994) have reported using the chromosomal DNA of a Mycobacterium paratuberculosis strain to construct an expression genomic library in E. coli. A recombinant clone, p35, that expresses a protein of approximately 35K was identified, and its gene product was used in the serodiagnosis of Johne's disease by immunoblotting. EI-Zaatari et al. reported a diagnostic yield with this clone that was higher than those reported using commercially available diagnostic tests. They suggest that the high sensitivity and specificity of p35 indicates a potential utility for the diagnosis of animals at all stages of Johne's disease.

EI-Zaatari et al. also tested the clone's potential as a probe, and found that it hybridized specifically to nine bacterial strains representing the Mycobacterium avium complex species and none of the other mycobacterial species or other related and unrelated bacteria.

Recently EI-Zaatari et al. (U.S. Pat. No. 5,776,692) have reported a recombinant clone pMptb #48 that expresses a 36K M. paratuberculosis antigen and its use as a test, as well as a mycobacterial genus-specific DNA probe corresponding to a 1.4 kb BamH1-DNA insert in pMptb #48. Serological tests are suggested for using the clone and/or the p36 k protein or fragments thereof.

The present inventor has also provided a serological composition and method for screening for an infection caused by Mycobacterium avium, such as Crohn's disease that have improved specificity and sensitivity and do not require a surgical procedure to obtain a test sample (U.S. Pat. No. 6,297,015, the disclosure of which is hereby incorporated hereinto by reference). This invention also provides a composition and method for predicting a predisposition to Crohn's disease and further for treating patients shown by the screening method to be infected with Mycobacterium paratuberculosis.

It is known in the art to prescribe specific dietary regimens for patients having gastrointestinal problems. For example, it is known to limit dairy and starch intake in patients with Crohn's disease (Gottschall, Ending the Vicious Cycle) and also to add probiotics to the diet to maintain and restore a healthy balance in the digestive system, especially to counteract negative effects of antibiotic therapy.

### SUMMARY OF THE INVENTION

It is therefore an object of the present invention to provide a method for treating an infection caused by Mycobacterium avium, such as Crohn's disease.

It is an additional object to provide such a method that has improved specificity and sensitivity over prior known compositions and methods.

Another object is to provide such a composition and method that do not rely on the use of corticosteroids.

It is also an object to provide such a composition and method that cause remission and improve the quality of life of a patient having Crohn's disease.

These objects and others are attained by the present invention, a method for detecting and treating a M. para. infection such as Crohn's disease in a human and for predicting a genetic predisposition thereto.

In an embodiment of the treatment method, the effective antibiotic is administered to a patient having been found positive for M. paratuberculosis by the serologic method of the invention. The treatment is supplemented by a diet including a regimen of probiotics and a specific carbohydrate diet.

In another embodiment of the method of the present invention, Crohn's disease is screened for by performing an ELISA analysis forserum antibodies to Mycobacterium avium subspecies paratuberculosis (MAP). For patients screening positive for MAP, a regimen of an antibiotic effective in and sufficient for eradicating a presence of Mycobacterium paratuberculosis is administered.

The features that characterize the invention, both as to organization and method of operation, together with further objects and advantages thereof, will be better understood from the following description.

### DETAILED DESCRIPTION OF THE PREFERRED EMBODIMENTS

A description of the preferred embodiments of the present invention will now be presented.

The p35 and p36 recombinant clones were used to perform humoral response testing to confirm M. paratuberculosis (also referred to in the literature as Mycobacterium avium ss. paratuberculosis, or MAP) infection by detecting the presence of specific antibodies in a patient. Although a positive response does not by itself indicate active infection, the quantitative measure of antibody titre supported by cutoff values may indicate infection activity levels. Serological testing requires only blood samples from subjects rather than the endoscopic or surgical procedures required to obtain tissue. Because false negatives can occur in BCG- and PPD-positive patients, patient history screening is necessary. Serological testing is thus believed to be a rapid and simple way to diagnose Crohn's disease.

The serological tests were based on the use of the two recombinant clones isolated from an M. paratuberculosis genomic library that expressed 35K and 36K MW antigens. Antigen p35 was isolated from Johne's disease sera (acid-fast bacilli form) and p36, from human CD sera (spheroplast form). The combined use of p35 and p36 recombinant antigens provides a highly specific and sensitive test to demonstrate the humoral immune response of CD patients to M. paratuberculosis.

Data were collected on 110 human sera, of which 63 were Crohn's patients and 47 controls (see Table 1). Among the controls were 35 volunteers with no history of GI tract disorder and 12 with ulcerative colitis. Subjects were free of tuberculosis and leprosy and had not received a bacillus calmet guerin (BCG)vaccination. The analysis was conducted using immunoblot against rabbit hyperimmune anti-M. para. antibodies. Of the 63 Crohn's sera tested, 49 (78%) reacted with p35, 57 (90%) with p36,48 (76%) with both antigens, and 58 (92%)with either antigen. A small portion of Crohn's samples, 5 (8%), did not react with one or both. Of the 35 sera from normal controls, none (0%) reacted with both antigens, 4 (11%) with p36,5 (14%) with p35, and 9 (25%) with either antigen. Of the 12 ulcerative colitis sera, only 1 (10%) reacted with p35 and/or p36, individually or combined.

TABLE 1
Serological Results

| | | +ve for | +ve for | +ve for either | +ve for both |
|---|---|---|---|---|---|
| | # tested | p35 | p36 | p35 or p36 | p35 and p36 |
| Crohn's disease | 63 | 45 (78%) | 57 (90%) | 58 (92%) | 48 (76%) |
| Ulcerative colitis | 12 | 1 (8%) | 1 (8%) | 1 (8%) | 1 (8%) |
| Normal controls | 35 | 5 (14%) | 4 (11%) | 9 (35%) | 0 (0%) |

These data suggest that using a combination of p35 and p36 antigens rather than individually is more specific for Crohn's disease diagnosis in an unexpectedly synergistic manner. Using both antigens does reduce the sensitivity of the assay but significantly increases the specificity. The data also confirm that there is a difference in reactivity between Crohn's samples and the controls at a reasonable level of significance (P<0.001) and further strengthens the association between Crohn's disease and M. paratuberculosis.

The data also support an improved serologic kit comprising the composition of the invention to provide earlier diagnosis and better treatment of Crohn's disease.

With the indication that Crohn's disease is at least in part caused by the presence of M. paratuberculosis, a treatment regiment including an administration of antituberculosis drugs was proposed. However, this bacterium is known to be resistant to most of these drugs. An in vitro study was performed to evaluate seven anti-TB drugs against M. para. isolated from resected tissue of CD patients using the Bactec system, which is known in the art, and the results are given in Table 2.

TABLE 2
Results of in vitro test of Seven Antituberculosis
Drugs against M. para.

| | RIF^{a} | ST^{b} | KM^{C} | CLR^{d} | INH^{e} | PZA^{f} | EMB^{g} | RIF:EMB | RIF:CLR |
|---|---|---|---|---|---|---|---|---|---|
| MIC₅₀ | >1.0 | >0.4 | <1.3 | <0.25 | >20 | >20 | <0.5 | <1.0:0.2 | <0.5:0.3 |
| (μg/ml)^{h} | | | | | | | | | |
| MIC_{99.9} | >2.6 | >3.0 | <3.0 | <1.25 | >20 | >20 | <3.0 | <1.0:1.0 | <0.5:1.2 |
| | | | | | | | | | or |
| Status^{j} | R | R | R | S | R | R | S | S | S |

^{a}Rifampicin; ^{b}streptomycin; ^{c}kanamycin; ^{d}clarithromycin; ^{e}isoniazid; ^{f}pyrazinamide; ^{g}ethambutol; ^{h}, i. 50, 99.9% M. para. cells inhibited or killed, resp.; ^{j}. R, resistant; S, sensitive.

Forty-two CD patients who tested serologically positive for M. para. were selected for rifabutin and macrolide antibiotic therapy (RMAT) for a duration of 6 to 22 months based upon their overall response to the treatment. Seven patients withdrew owing to their inability to tolerate the medications, leaving 35. The regimen included 250 mgm po bid clarithromycin, 150 mgm 1 po bid rifabutin, and 200 mgm po qd of a probiotic containing equal amounts of Lactobacillus acidophilus and Lactobacillus rhamnosus.

The patients were assessed to determine overall response to the treatment. 77.1% (27/35) of the patients achieved a state of clinical remission (as defined by the Crohn's Disease Activity Index, CDAI, criterion with a score<150) while being off all other medications, such as immunosuppressants and corticosteroids. The majority of these patients had acute presentation of CD when placed on RMAT. 8.6% (3/35) of the patients were partial responders, and 14.3% (5/35)were nonresponders. 16.7% (7/42)withdrew, as stated above, since they were unable to tolerate the RMAT medications and discontinued therapy. These findings support the use of RMAT in the treatment of CD.

This work has been published by the present inventor and colleagues [I. Shafran et al., “Open Clinical Trial of Rifabutin and Clarithromycin Therapy in Crohn's Disease,” Digestive and Liver Disease 34(1), 22-28, 2002]. The contents of this paper are incorporated herein by reference.

In a particular case study, a 65-year-old patient having been diagnosed with Crohn's disease at age 30 was found to be PCR positive for M. para. with humoral immune response against recombinant antigens of M. para. An endoscopy was performed through the patient's stoma and found a 4.0-cm aphthous ulcer. The remaining ileum was unremarkable to a depth of 120 cm. Histology indicated typical features of CD.

The patient demonstrated significant healing (80%) of an ulcer seen in the ileum by endoscopy following a regimen of 250 mg clarithromycin twice a day and 150 mg rifabutin daily. The patient became asymptomatic in 2 weeks, and a followup endoscopy was performed after completing 1 month of treatment. The 4-cm ulcer had reduced in size to 1 cm, with excellent reepitheliazation from the edge of the ulcer inward. The remaining ileum to 120 cm was normal. The patient has remained symptom-free and continues on the antibiotic regimen.

In further studies on fistula healing (I. Shafran, L. Kugler, and J. Sandoval, submitted to Gastroenterology), ten MAP-positive patients having acute lower gastrointestinal Crohn's disease with fistulization were identified. These patients failed to respond to prior corticosteroid and anti-inflammatory therapy and presented a mean CDAI score of 269, ranging from 250 to 300. RMAT therapy was given, with 250 mg 1 po bid clarithromycin and 150 mg 1 po bid rifabutin. Antibiotic therapy was complemented with 200 mg po bid of probiotic containing equal amounts of Lactobacillus acidophilus and Lactobacillus rhamnosus.

All patients exhibited complete closure of the fistuli within an average time span of 32 weeks of treatment and presented a mean of 126.5 points on a final CDAI score. All patients reached a state of remission as classically defined by a CDAI score below 150 points, with a minimum differential of 100 points from initial to final score. A recurrence of fistuli within an average time of 8 months occurred in 4 patients who elected to discontinue RMAT therapy.

As this study was continued, 35 patients with CD were being treated with RMAT. 37% (13/35) of the patients developed a serum sickness-like illness during the first 4-6 weeks of treatment. The patients experienced flu-like symptoms such as fever, chills, moderate to severe arthralgia, back pain, anorexia, and fatigue. These symptoms generally lasted for a full week and dissipated over the following 3 weeks. With each patient, a majority of symptoms stopped within the first month of treatment. It was also found that these symptoms responded well to Cox-2 inhibitors (celecoxib-200 mgm po qd) with no adverse effects or worsening of colitis noted during treatment. These observations suggest that the Cox-2 inhibitors may help in controlling the initial side effects of RMAT. It is also thought that this serum sickness may be a Jarisch-Herxheimer reaction in response to the antimicrobial therapy.

Current hypotheses are being investigated regarding the causative agent(s) of Crohn's disease. While many workers in the field have become convinced of the involvement of M. para., it may well turn out that this bacterium is but one of a number of pathogenic agents. Therefore, the regimen proposed herein preselects patients for antibiotic treatment by the detecting method of the present invention, the combined p35/p36 serological test, patients testing negative for M. para. being less likely to experience alleviation of CD symptoms under the antibiotic regimen.

In a further study, 120 patients with Crohn's disease whose sera tested positive to MAP were selected for treatment with rifabutin and macrolide therapy (RMAT), here 150 mg rifabutin twice a day and 500 mg clarithromycin daily. Probiotic supplements were also given, here comprising 500 million units lactobacillus acidophilus, 500 million units of bifidobacterium bifidum, and a combination of one billion units of bifidobacterium longum, infantis, rhamnosus, and lactobacillus salivarius, reuteri, casei, bulgaricus, sporogenes, laterosporus, and plantarum. It is believed that this probiotic supplementation counterbalances antibiotic-induced degradation of intestinal probiotic flora.

Also effective is an administration of a “specific carbohydrate diet,” herein defined substantially in accordance with the teachings of E. Gottschall (Breaking the Vicious Cycle, Kirkton Press Ltd., Ontario, Canada, 1994; the contents of this volume are incorporated herein by reference). This regimen has been proven effective in alleviating intestinal symptoms associated with Crohn's disease. Briefly, the diet comprises no food containing carbohydrates other than those found in fruits, honey, yoghurt having substantially all lactose digested, and vegetables and nuts contained in a listed group.

The patients' response to treatment was monitored over a period of 36 months. 20 patients withdrew from study owing to intolerance to side effects of the medications. Of the remaining 100 patients, as of the time of writing, 62 have completed 1 year of treatment; 28, 2 years; and 10 patients, in the third year.

A significant response is traditionally defined as a reduction in the CDAI score of 70 points between entrance and exit of a program, as well as an absence of a need for other medication. In this study the overall response to treatment was 75% at the end of 2 years, with 63 of 90 patients in complete remission after 1 and 2 years of treatment. The patients in their third year of treatment are all in remission. Of the nonresponding patients (27 total), 12 required surgical intervention for obstruction and 15 are on immunotherapy and remain in evaluation.

Another study was undertaken to compare age-matched MAP-positive and -negative patients utilizing ELISA analysis forserum antibodies to MAP. 10 MAP-positive and 10 MAP-negative patients were compared at 1 year, utilizing the CDAI score as a primary endpoint. The elimination of corticosteroids and the need for immunotherapy were considered secondary endpoints. The MAP-positive patients achieved complete remission in 9 out of the 10 cases, with entry CDAI score of 285 and exit score of 111. Steroids were eliminated in 8 of 10 patients, with 2 patients requiring low-dose steroids. In the MAP-negative group at 1 year, 6 of 10 patients were in complete remission, with an entry CDAI score of 308 and an exit score of 179. Of the responsive patients, 3 of 6 required additional immunotherapy, and 3 remained on antibiotics alone. In the unresponsive patients, infliximab was used with induction of remission in all patients.

These data suggest a predictive value for the MAP ELISA test to determine who may be best suited for antibiotic therapy. Further studies have been undertaken using this marker to evaluate ELISA response over time in CD patients treated with antibiotics.

It may be appreciated by one skilled in the art that additional embodiments may be contemplated, including other recombinant clones chosen from the M. paratuberculosis genomic library and other antibiotic regimens for the treatment of bacteria-positive CD patients.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the compositions and methods illustrated and described herein are by way of example, and the scope of the invention is not limited to the exact details of structure, synthesis, and delivery.

What is claimed is:

1. A method for treating a human patient suspected of having Crohn's disease comprising the steps of:
screening for Crohn's disease by:
simultaneously contacting a human serum sample with an antigen composition comprising a 35 kD protein expressed by a recombinant p35 clone specific to sera from Johne's disease and a 36 kD protein expressed by a recombinant p36 clone specific to sera from Crohn's disease;
detecting a bound antibody-antigen complex to the antigen composition, wherein the bound antibody-antigen complex detects a presence of Mycobacterium avium ss. paratuberculosis, and thus indicates a presence of Crohn's disease;
administering a regimen of an antibiotic effective in and sufficient for eradicating a presence of Mycobacterium paratuberculosis; and
administering a regimen of a probiotic and a specific carbohydrate diet.

2. The method recited in claim 1, wherein the detecting step comprises conducting an immunoblot test against rabbit hyperimmune anti-Mycobacterium paratuberculosis antibody.

3. The method recited in claim 1, wherein the antibiotic comprises a combination of rifabutin and clarithromycin.

4. The method recited in claim 3, wherein the rifabutin is administered in a dosage of 150 mg twice daily and the clarithromycin is administered in a dosage of 500 daily.

5. The method recited in claim 1, wherein the probiotic comprises Lactobacillus acidophilus and Lactobacillus rhamnosus.

6. The method recited in claim 5, wherein the Lactobacillus acidophilus and Lactobacillus rhamnosus are administered in substantially equal amounts of 200 mgm po qd.

7. The method recited in claim 1, wherein the antibiotic comprises at least one of clarithromycin or ethambutol.

8. The method recited in claim 1, wherein the antibiotic comprises a combination of rifampicin and ethambutol.

9. The method recited in claim 1, further comprising the steps, following the administering step, of:
determining whether a treated patient is experiencing a serum sickness-like illness; and
if the determining step is positive, treating the patient with a Cox-2 inhibitor.

10. The method recited in claim 9, wherein the Cox-2 inhibitor comprises celecoxib.

11. The method recited in claim 10, wherein the celecoxib is administered in an oral dose of 200 mgm once per day.

12. A method for treating a human patient suspected of having Crohn's disease comprising the steps of:
screening for Crohn's disease by performing an ELISA analysis for serum antibodies to Mycobacterium avium subspecies paratuberculosis (MAP); and
for patients screening positive for MAP, administering a regimen of an antibiotic effective in and sufficient for eradicating a presence of Mycobacterium paratuberculosis.

13. The method recited in claim 12, further comprising the step of administering a regimen of a probiotic.

14. The method recited in claim 13, wherein the probiotic comprises Lactobacillus acidophilus and Lactobacillus rhamnosus.

15. The method recited in claim 14, wherein the Lactobacillus acidophilus and Lactobacillus rhamnosus are administered in substantially equal amounts of 200 mgm po qd.

16. The method recited in claim 13, wherein the probiotic comprises 500 million units lactobacillus acidophilus, 500 million units of bifidobacterium bifidum, and a combination of one billion units of bifidobacterium longum, infantis, rhamnosus, and lactobacillus salivarius, reuteri, casei, bulgaricus, sporogenes, laterosporus, and plantarum.

17. The method recited in claim 13, further comprising the step of administering a regimen comprising a specific carbohydrate diet.

18. The method recited in claim 12, wherein the antibiotic comprises a combination of rifabutin and clarithromycin.

19. The method recited in claim 18, wherein the rifabutin is administered in a dosage of 150 mg twice daily and the clarithromycin is administered in a dosage of 500 daily.
